(19) United States
(12) Patent Application Publication
(10) Pub. No.: US 2011/0004053 A1
(43) Pub. Date: Jan. 6, 2011

(54) BOLSTER FOR SECURING A SEPTAL SPLINT TO A CARDIAC WALL, A METHOD OF USE THEREOF, AND A SYSTEM INCLUDING THE SAME

(75) Inventors: David B. Melvin, Loveland, OH (US); Sue P. Melvin, Loveland, OH (US)

Correspondence Address: WOOD, HERRON & EVANS, LLP, 2700 CAREW TOWER, 441 VINE STREET, CINCINNATI, OH, 45202, US

(73) Assignee: SURGICAL ENERGETICS, INC., Cincinnati, OH (US)

(21) Appl. No.: 12/746,284

(22) PCT Filed: Dec. 5, 2008
(86) PCT No.: PCT/US08/85693

§ 371 Date: Sep. 15, 2010

Related U.S. Application Data
(60) Provisional application No. 60/992,639, filed on Dec. 5, 2007.

(51) Int. Cl. A61F 2/02

(52) U.S. Cl. 600/37

(57) ABSTRACT

The invention is generally concerned with supporting the ventricular septum (9) of the heart during mechanical compression of at least one ventricle. The ventricular septum (9) is supported by a septal splint (1) coupled by at least one bolster (48) to a free-wall actuating mechanism (6). The septal splint (1) has a central patch (2) with radial strands (3) extending to the junction of the free wall (7) and septum (9). The radial strands (3) traverse the ventricular free wall (7) at the junction. Affixation of each strand (3) to the heart wall is controlled by an internal element (18, 21) and external element (19, 38) of the bolster (48) and a placement tool (46) to avoid risks of injuring major coronary blood vessels. The placement tool (46) is configured to hold, deliver, stabilize for passage, and release the internal and external elements (18, 21, 19, 38) of the bolster (48), along with accompanying radial strands (3) from the septal splint (1).

### RELATED APPLICATIONS

This Application is a U.S. National Phase of PCT/US2008/085693 entitled “A Bolster for Securing a Septal Splint to a Cardiac Wall, a Method of Use Thereof, and a System Including the Same” filed Dec. 5, 2008, which claims the benefit of U.S. Provisional Application Ser. No. 60/992,639 entitled “Septal Splint With Precisely Controlled, Bolster-Supported, Transmural Fixation” filed Dec. 5, 2007, the disclosures of which are hereby incorporated by reference herein.

### FIELD OF THE INVENTION

This invention relates generally to mechanical actuation of the cardiac walls, and more specifically to a bolster for securing a septal splint to a cardiac wall, a method of use thereof, and a system including the same, such as for use in actuation of one or more cardiac walls.

### BACKGROUND OF THE INVENTION

The deadly deficiency of heart failure is that the heart is no longer capable of pumping blood at sufficient levels to perfuse the tissues of the body. And, mechanical blood pumps have not solved the problems associated with heart failure. Despite mechanical effectiveness, each type of pump currently in use, whether continuous or pulsatile, carries the risk of inducing both immunologic and thrombotic compromise such that only a tiny, most desperate fraction of all the heart failure sufferers are referred for, and are helped by, any of them. The evidence suggests that these disastrous events are caused by the never-healing, artificial blood-contacting surfaces of the mechanical blood pumping devices. The non-reactive nature of native intact endothelium is frustratingly difficult to recreate with the synthetic materials used in these devices.

By way of example, reciprocating pumps produce pulsatile flow using a continuously flexing diaphragm. These diaphragms are produced from both smooth and textured polyurethanes. Texturing the diaphragm may lessen the risk of embolization while increasing the risk of immune sensitization and membrane failure. Moreover, none of the current pulsatile pumps has a durability specification beyond five years. With regard to rotary pumps, despite steady decreases in shear damage and thrombi, these pumps are still troubled by reports of both thromboembolism and immune activation.

Global heart-wall actuation is an alternative to the blood pumps discussed above that circumvents blood contact with synthetic surfaces. However, global heart-wall actuation has only succeeded in either short-term resuscitation or longer supplemental ‘boosting’. The attraction of this approach is that the true deficit—power—is addressed without replacing endocardium and thereby decreasing the risks of immunologic and thrombotic compromise.

Efforts began in the 1950s to directly and physically restore heart wall motion by applying mechanical forces to the walls of the heart. However, existing devices compress, either directly or indirectly, both ventricles together, forgoing pressure independence of the right and left ventricles. Pressure independence of the right and left ventricles is essential to maintaining the greatly different arterial pressures in the pulmonary and systemic circulatory systems, respectively. Compromising pressure independence limits the use of existing compression devices to either brief use or modest supplementation.

For example, Bencini supplemented heart function by tidally infusing and withdrawing pericardial fluid. Vineberg tried a rhythmically inflatable heart-jacket. Both Jones and Rosenberg used a localized intrapericardial balloon. Kolobow and Bowman applied a suction-expanded rubber heart jacket, which was cyclically allowed to recoil. The most successful was invented by Dr. George Anstadt in 1965 and consisted of a glass cup held on the two ventricles by apical suction. Alternate air pressure and vacuum were applied to a polymer lining in the glass cup. This apparatus restored cardiac output, surpassed closed-chest resuscitation, and bridged to transplant.

There have been at least three serious recent development projects toward ventricular actuation. AbioMed, Inc.'s ‘Abiobooster’ is a multi-chambered pneumatic jacket. And, CardioTechnologies, Inc. and MyoTech have modified and softened the Anstadt Cup. Each of these devices has generated promising experimental results with goals of supplementing cardiac contraction. And, each of these devices supplements cardiac contraction using global compression of the heart. Global compression is characterized by the absence of reliable pressure isolation of left and right ventricles, which poses risks of obliterating the lower-pressured right ventricle with inevitable septal-free wall forceful impact in each systole if the devices are used for more than just ‘boosting’. The septum-stabilizing assist device taught in U.S. Pat. No. 5,957,977 addresses this problem.

The technology in the U.S. Pat. No. 5,957,977 requires puncture of the right ventricular free-wall to connect an internal septal support to an outside actuator or to an intermediate framework structure. Penetrating the ventricular free wall creates risks of (1) coronary artery or cardiac vein injury by the traversing suture or other tensile element and (2) erosion of or bleeding along the track of the traversing tensile member. One aspect of the present invention addresses safety from vessel injury by precise control of the site of wall penetration. Another aspect of the invention addresses bleeding and erosion by utilizing a coupling material shown to securely integrate with muscular tissue in a wide range of animal models.

### SUMMARY OF THE INVENTION

In accordance with an embodiment of the invention, a bolster is provided that includes an external element defining an elongated body member having at least one aperture extending therethrough. The elongated body member is configured for placement adjacent an external surface of a cardiac wall. The bolster further includes an internal element that includes a free wall leaf and a septal leaf joined together to define a body member. The free wall leaf has at least one aperture extending therethrough. The body member is configured for placement inside a cavity of the heart. And, the external element and the internal element are configured for coupling to each other across the cardiac wall via the apertures.

In accordance with another embodiment of the invention, a system is provided for assisting the function of a natural heart. The system includes a septal splint configured for use in assisting the function of a natural heart and at least one bolster configured to secure the septal splint to a cardiac wall. The bolster includes an external element defining an elongated body member having at least one aperture extending therethrough. The elongated body member is configured for placement adjacent an external surface of a cardiac wall. The bolster further includes an internal element that includes a free wall leaf and a septal leaf joined together to define a body member. The free wall leaf has at least one aperture extending therethrough. The body member is configured for placement inside a cavity of the heart. And, the external element and the internal element are configured for coupling to each other across the cardiac wall via the apertures.

In accordance with yet another embodiment of the invention, a method is provided for assisting the function of a natural heart. The method includes securing at least one bolster to a cardiac wall. The bolster includes an external element defining an elongated body member having at least one aperture extending therethrough. The elongated body member is configured for placement adjacent an external surface of a cardiac wall. The bolster further includes an internal element that includes a free wall leaf and a septal leaf joined together to define a body member. The free wall leaf has at least one aperture extending therethrough. The body member is configured for placement inside a cavity of the heart. And, the external element and the internal element are configured for coupling to each other across the cardiac wall via the apertures. A septal splint is secured to the cardiac wall via the bolster, with the septal splint being situated in the right ventricle of the heart. And, a free-wall actuating mechanism is coupled to the septal splint via the bolster, with the free-wall actuating mechanism being positioned over the left ventricle of the heart.

By virtue of the foregoing, there is thus provided a bolster for securing a septal splint to a cardiac wall, a method of use thereof, and a system including the same.

### BRIEF DESCRIPTION OF THE DRAWINGS

The accompanying drawings, which are incorporated in and constitute a part of this specification, illustrate embodiments of the invention and, together with a general description of the invention given above, and the detailed description of the embodiments given below, serve to explain the principles of the invention.

FIG. 1 is a perspective view of an embodiment of a septal splint;

FIG. 2 is a perspective view of the septal splint of FIG. 1 further including a mitral annular ring;

FIG. 3 is a perspective view of the septal splint with mitral annular valve of FIG. 2 further including a passive sleeve and a free-wall actuating mechanism;

FIG. 4 is a split view showing, on the left-hand side, a region of a radial strand of the septal splint of FIG. 1 and, on the right-hand side, a photomicrograph of tissue ingrowth into such a strand;

FIG. 5 is a translucent view of a heart with the device of FIG. 3;

FIG. 6 is a transverse sectional view through the ventricular septum and left and right ventricular cavities of a sheep heart;

FIG. 7 is a diagrammatic view of a transverse section through the ventricular septum and left and right ventricular cavities of a heart;

FIG. 8A is a perspective view of an embodiment of a bolster for securing a septal splint in accordance with the present invention;

FIG. 8B is a cross-sectional view of the bolster of FIG. 8a secured in place to the heart wall via a rigid moment-sustaining element;

FIG. 8C is a cross-sectional view of the bolster of FIG. 8a secured in place to the heart wall via both a compressive moment-sustaining element and a tensile element;

FIG. 9 illustrates a method for reducing ventricular tension by altering the shape of the left ventricle;

FIG. 10 is a perspective view of an embodiment of an inner element of a bolster for use in securing a septal splint in accordance with the present invention;

FIG. 11 is a perspective view of the inner element of FIG. 10 mounted on a placement tool;

FIG. 12 is an enlarged perspective view of the placement tool of FIG. 11;

FIG. 13 is a perspective view of an embodiment of an outer element of a bolster for use in securing a septal splint in accordance with the present invention mounted on an opposite end of the placement tool of FIG. 11;

FIGS. 14-16B show the placement tool in use, i.e., the positioning of and relationship between the inner and outer elements of the bolster for securing in place thereof to the heart wall;

FIG. 17A is an enlarged perspective view of a portion of the placement tool of FIG. 12 with guide wires attached to radial strands being fed through the tool;

FIG. 17B-17C illustrate the guide wires and radial strands of FIG. 17A being fed through the internal element of the bolster, the free wall of the right ventricle, and the external element of the bolster;

FIG. 18 illustrates releasing the internal and external elements and the removal of the placement tool from within the heart after tying off the radial strands; and

FIG. 19 is a cross-sectional view of the right ventricle and a portion of the left ventricle showing the coupling of the septal splint to a free-wall actuating mechanism via inner and outer elements.

### DETAILED DESCRIPTION

One or more specific embodiments of the present invention will be described further below. In an effort to provide a concise description of these embodiments, all features of an actual implementation may not be described in the specification. It should be appreciated that in the development of any such actual implementation numerous implementation-specific decisions must be made to achieve the developers' specific goals, which may vary from one implementation to another. Moreover, it should be appreciated that such a development effort might be complex and time consuming, but would nevertheless be a routine undertaking for those of ordinary skill having the benefit of this disclosure.

When introducing elements of the present invention (e.g., the exemplary embodiments(s) thereof), the articles “a”, “an”, “the” and “said” are intended to mean that there are one or more of the elements. The terms “comprising”, “including” and “having” are intended to be inclusive and mean that there may be additional elements other than the listed elements.

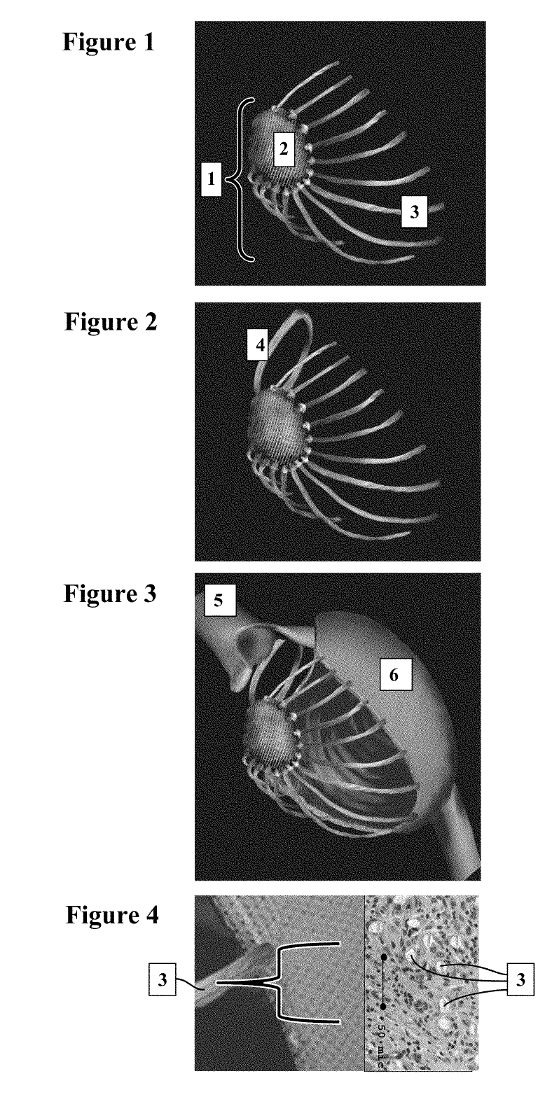

FIGS. 1-19 disclose aspects of the present invention, which are discussed in more detail below, yet relate generally to a bolster (48) for coupling a septal splint (1) to a free-wall actuating mechanism (6) to provide support for the ventricular septum (9) during operation of the actuating mechanism (6). The bolster (48) includes an internal element (18 or 21) and an external element (19 or 38). The internal element (18, 21) is generally implanted in the right ventricle (10) along the intersection of the right ventricle free wall (7) and the ventricular septum (9). The external element (19, 38) is placed adjacent the external surface of the heart in at least partial alignment with the internal element (18, 21). The internal and external elements (18, 21, 19, 38) are coupled to each other by at least one of a rigid element (15), a compressive element (16), and/or a tensile element. The tensile element may be a portion of the septal splint (1), i.e., the radial strand (3). The free-wall actuating mechanism (6) is coupled to the external element (19, 38) such that compressive forces generated by the actuating mechanism (6) are transferred to the ventricular septum (9) by the bolsters (48) and septal splint (1).

FIG. 1 illustrates a septal splint (1) assembled alone with its components being the central portion (2) and a plurality of radial strands (3).

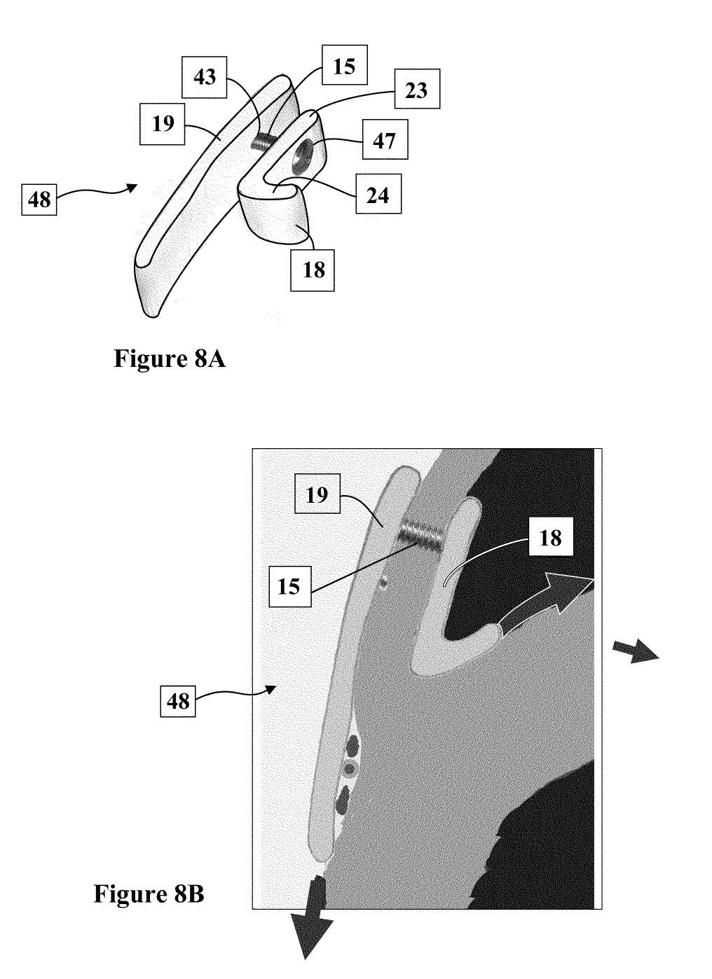

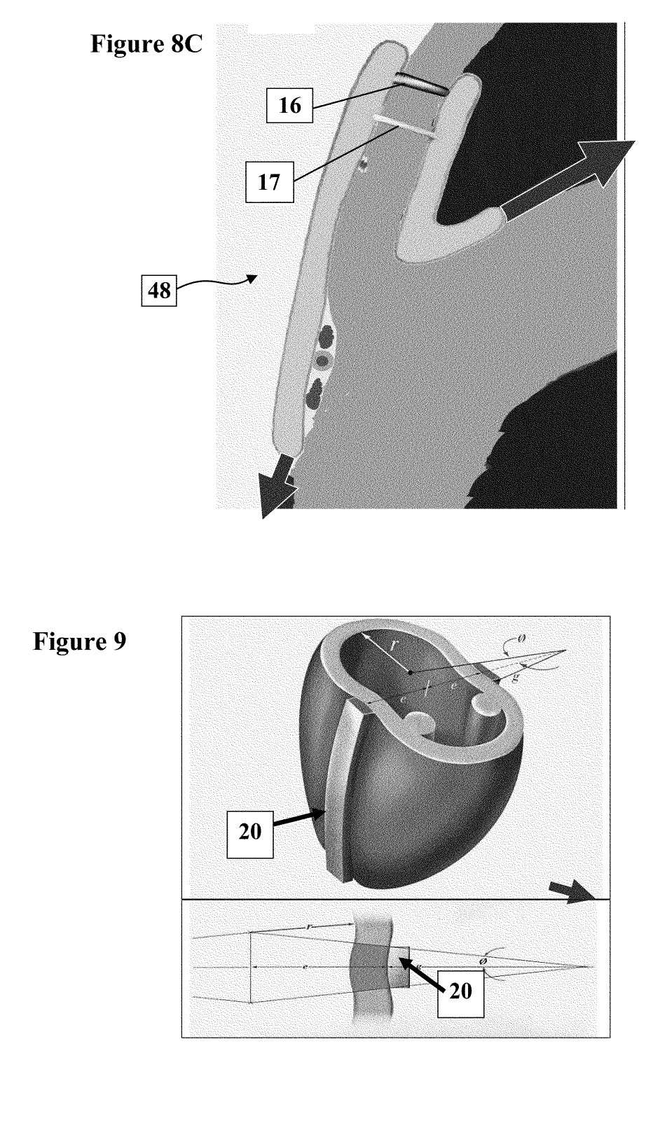

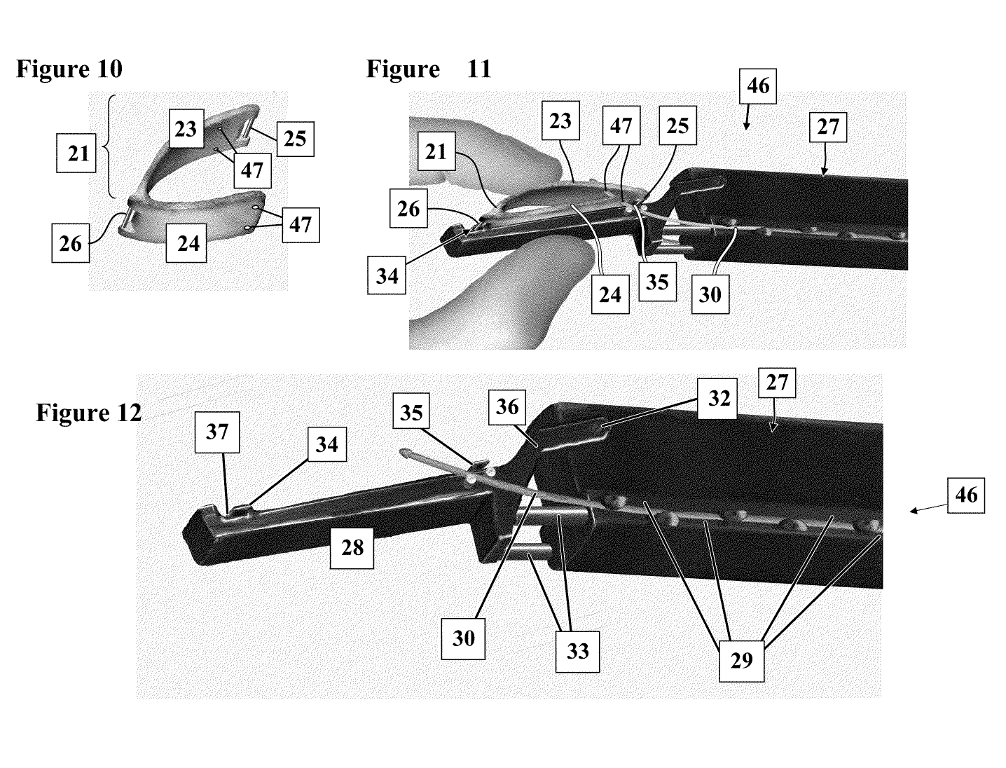

The central portion (2) functions as a central anchoring point for the radial strands (3). In one embodiment, the central portion (2) may be a patch of material to which the radial strands (3) are coupled. In this embodiment, the proximal end of the radial strands (3) attaches to the central portion (2). In another embodiment, the radial strands (3) are continuous extensions of the central portion (2). The distal ends of the radial strands (3) are configured for coupling to an extracardiac free-wall actuating mechanism (6) (FIG. 3). In one aspect of the invention, the distal ends of the radial strands (3) are coupled to the extracardiac free-wall actuating mechanism (6) by one or more bolsters (48) (FIGS. 8A and 10), as is described in greater detail herein below. Examples of free-wall actuating mechanisms suitable for use in the present invention are more fully described in U.S. Pat. No. 5,957,977 and U.S. Publication Nos. 2006/0155160 and 2006/0187550, each of which are incorporated by reference herein in their entirety. The coupling of the radial strand (3) to the central portion (2), the bolsters (48), and/or the extracardiac free-wall actuating mechanism (6) may be direct or indirect.

The central portion (2) and radial strands (3) may be any biocompatible material suitable for use in the vascular system. Non-limiting examples of suitable materials are braided polyester suture, ePTFE (expanded polytetraflurethylene), or braided polyester ribbon. In addition, the central portion (2) may be a textile or membranous material.

FIG. 2 shows the septal splint (1) configured with a mitral annular ring (4) that functions as an additional internal support structure.

FIG. 3 shows the septal splint (1) with mitral annular ring (4), and two extracardiac components: a passive sleeve (5) to support the aortic root and proximal coronary arteries, and the free-wall actuating mechanism (6). In this embodiment, the distal ends of the radial strands (3) are shown coupled to the free-wall actuating mechanism (6).

FIG. 4 is a split view showing the region of one radial strand (3) of the septal splint (1) which traverses the free ventricular wall and also a photomicrograph of tissue integration of the same type of strand (3), which is a polyester fine-fiber bundle, used in the septal splint (1) after 60 days of healing in a muscle. The septal splint (1) may undergo endothelialization wherein the native heart lining tissue tends to grow over and anchor to any porous material that is not excessively flexing.

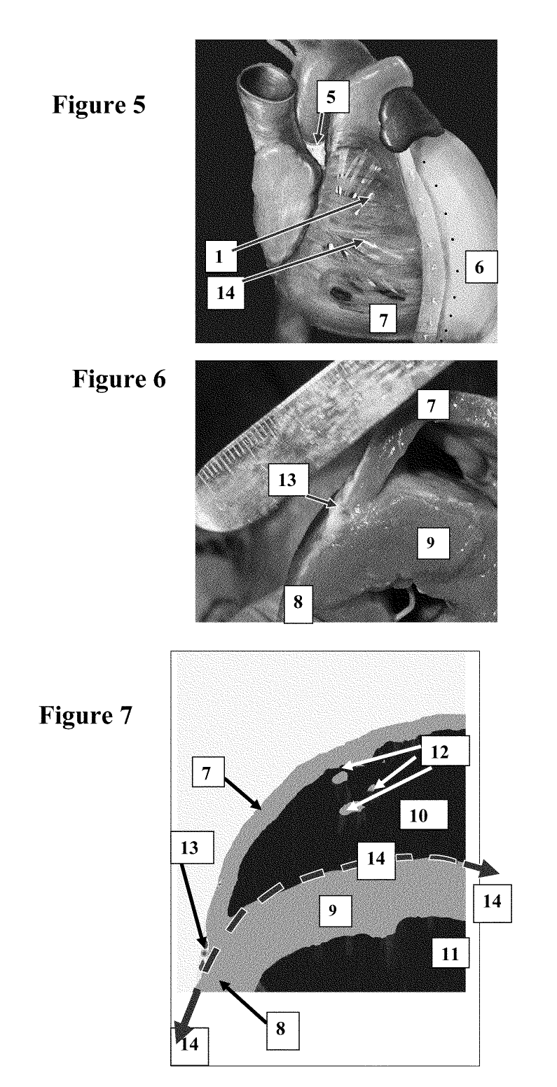

FIG. 5 is a translucent view of the heart with the septal splint (1) and the other associated components illustrated in FIG. 3. In practice, only the sleeve (5) and the actuating mechanism (6) are normally visible outside the heart. For orientation purposes, the septal splint (1) is illustrated in this figure by being revealed through a translucent anterior right ventricular wall (7).

FIG. 6 is a photograph of a cut sheep heart showing a transverse section through the ventricular septum (9) and the adjacent part of the left (11) and right (10) ventricular cavities. The coronary arteries (13) and cardiac veins of greatest vulnerability to damage with septal reinforcement techniques run subepicardially along the anterior and posterior interventricular grooves (anterior shown here). The devices and techniques described herein decrease the risk of damage to these blood vessels that is generally associated with septal reinforcement techniques.

FIG. 7 is a diagrammatic transverse section through the ventricular septum (9) and the adjacent part of the left (11) and right (10) ventricular cavities. The coronary arteries (13) and cardiac veins run subepicardialy along the anterior and posterior interventricular grooves (anterior shown here). As illustrated in FIG. 6, these vessels are in an area that is a very likely penetration point for a needle or other device that penetrates the right ventricular free wall (7) at the junction of the right ventricular septum (9) and the right ventricular free wall (7), such as the radial strands (3) of the septal splint (1). As further discussed in more detail below, the radial strands (3) are advanced along the right ventricular septum (9), between that septum and intracavitary structures such as papillary muscles and chordae tendinae (12), and are pushed through the right ventricle's freewall (7) near the free-wall/septal junction, with the aim of continuation tangent with the left ventricle's freewall (8). Septal support strands, such as the radial strands (3) of the septal splint (1), advance over this path because the lowest-energy path for such flexible constricting tensile element, which surrounds the left ventricle (11) and its walls (8, 9), tends to be a circular path, as represented generally by the dashed lines. However, traversing a needle attached to a radial strand (3) through the wall of the right ventricle following this path is risky because critically important coronary arteries (13) and coronary veins are very near that path.

FIGS. 8A-20 generally illustrate devices and methods to avoid an unsafe passage of tensile elements, such as radial strands (3) of the septal splint (1) directly through vital regions of coronary arteries and veins of the heart by using at least one bolster (48). The bolsters (48) have two components, external element (19 or 38) and internal element (18 or 21). The external element (19, 38) is configured for placement adjacent an external surface of a wall of the heart. The internal element (18, 21) is configured for placement inside a cavity of the heart, such as the right ventricle, and in at least partial alignment with the external element (19, 38). The external element (19, 38) and internal element (18, 21) are also configured for coupling to each other across the heart wall.

FIGS. 8A and 8B illustrate one embodiment of the bolster (48) in which the bolster (48) is rigid and has a cantilevering action. This bolster (48) uses moment-sustaining members, i.e., rigid internal and external elements (18, 19), inside and outside the heart for coupling thereof together, so that contractile force generated by the free-wall actuating mechanism (6) is transmitted to a site somewhat removed from the coronary blood vessels described above. The site of force transmission is controllable by device selection and positioning.

With specific reference to FIG. 8A, in this embodiment, the internal element (18) and external element (19) can be coupled together by a moment sustaining rigid element (15) and possibly a tensile element (not shown), e.g., the free end of the radial strands (3) of the septal splint (1). The rigid element (15) may be any suitable moment sustaining form, such as a threaded bolt, as is illustrated in FIGS. 8A and 8B. The external element (19) defines an elongated body member, which includes at least one aperture (43) extending therethrough for receiving the moment sustaining rigid element (15). The internal element (18) defines a body member that includes a free wall leaf (23) and a septal leaf (24) joined together. The free wall leaf (23) includes at least one aperture (47) extending therethrough for receiving the moment sustaining rigid element (15). While other configurations are contemplated, the internal element (18) is shown curved. Accordingly, the external element (19) and the internal element (18) are configured for coupling to each other across the cardiac wall via the apertures (43, 47).

FIG. 8B more clearly shows the bolster (48) with the internal element (18) positioned inside a cavity of the heart, i.e., the right ventricle, and the external element (19) positioned adjacent an external surface of the cardiac wall. The bolster (48) has rigid moment-sustaining element (15) across the heart wall to couple the internal and external elements (18, 19) together. The rigid moment sustaining element (15) functions to directly transmit from external element (19) to the internal element (18) the force generated by the free-wall actuating mechanism (6), as discussed further below.

FIG. 8C shows a moment-sustaining combination of at least one essentially compressive element (16) and tensile element, e.g., radial strands (3), that traverses the heart wall between the internal and external elements (18, 19). A non-limiting example of a compressive element (16) is a pin. Another exemplary tensile element, other than the radial strands (3), is a suture (not shown). For similar moment sustaining ability of the wall-crossing components, both mass and volume of implanted material and caliber of puncture wound in the heart wall tissue should be far less than with a penetrating rigid member (15) as in FIGS. 8A and 8B. The bolster (48), which is cantilevered, is made from a rigid biocompatible material, such as plastics, ceramics, glasses, composites and metals, such as titanium and titanium alloys.

The bolster elements (18, 19) may also be padded with one or more layers of biocompatible surface materials such as textile fabrics and polymeric membranes. The bolsters (48) may also allow for tissue ingrowth, including endothelialization, at their surfaces and may optionally be treated with structural modifications and or biologically active agents to promote tissue ingrowth.

Another method to safely traverse the heart wall in areas heavily populated with coronary vessels is by using a less rigid/resilient type of bolster (48) which allows the displacement of the areas of heart wall heavily populated by coronary vessels. Bolsters (48) that are resilient may reduce the risk of tissue erosion and trauma to the heart wall. These types of bolsters (48) preclude the cantilevering action of the embodiments of FIGS. 8a-8c and require that force be returned to its lowest energy state, an essentially circular arc in its passive regions. This can be done safely if the regions of heart wall heavily populated by coronary vessels are inwardly displaced.

FIG. 9 refers to computational and experimental work showing that altering ventricular shape to reduce ventricular tension can be safely done. Both computational and experimental evidence from at least two approaches to tension-reducing ventricular shape alteration have confirmed that localized external forces, if a force applicator (20) has smooth edges, are well tolerated whether the force/displacement applicator is widely distributed (the CardioClasp experience with bars (20) supported at the cardiac circumference as shown) or more narrowly concentrated (the MyoSplint experience with buttons a few millimeters wide, tethered across the ventricular cavity).

FIG. 10 shows a less rigid or resilient type of internal element (21) to facilitate altering ventricular shape to reduce tension. The internal element (21) defines a body member having a free-wall leaf (23) and septal leaf (24) joined together along an edge. The free wall leaf (23) and the septal leaf (24) include at least one aperture (47) extending therethrough. The internal element (21) may be made of resilient biocompatible materials having the desired resilient properties, such as a metal framework of stainless steel or other biocompatible metal, and/or a low durometer silicone rubber sandwiched between layers of polyester cloth such as a double knit. Resilient is understood to mean that the free-wall leaf (23) and the septal leaf (24) may return to their original relative positions after being collapsed. While other configurations are contemplated, the internal element (21) is shown curved. The framework of the free wall leaf (23) may be continuous with optional proximal (25) and distal (26) end pins, each approximately 0.040″ (1 mm) in diameter, for purposes later discussed.

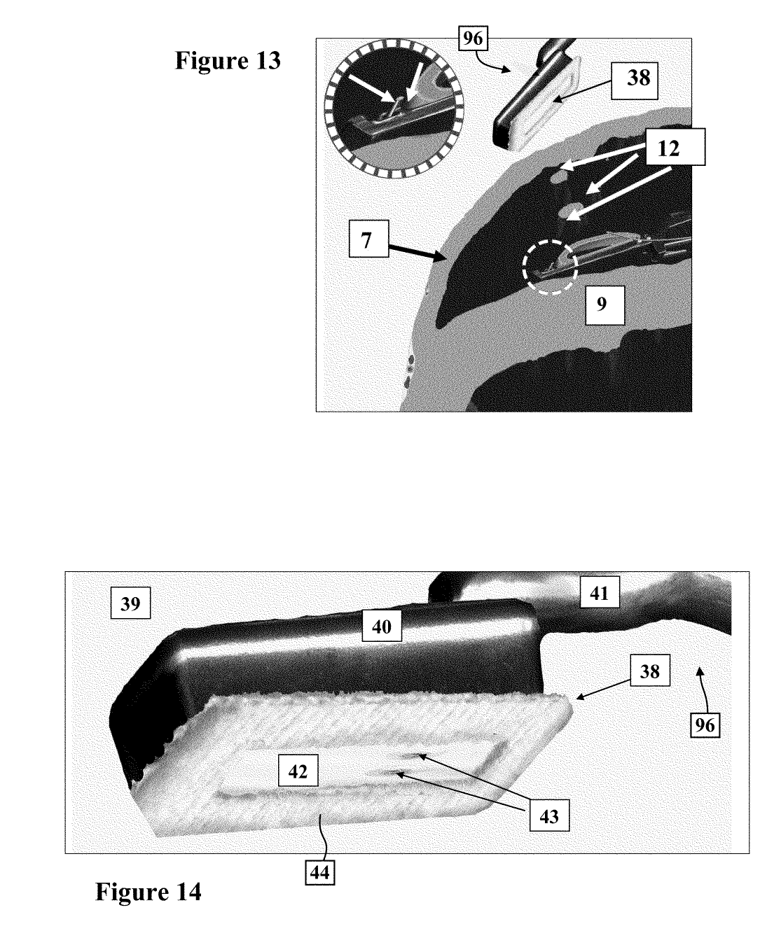

FIG. 11 illustrates internal element (21) mounted on pivoting tray (28) of an inner jaw (27) of a placement tool (46) prior to insertion into the right ventricular cavity of the heart. The placement tool (46) has inner jaw (27) for placing the internal element (21) in the cavity of the right ventricle and an outer jaw (39) (FIG. 13) for placement of the external element (38) adjacent an external surface of a cardiac wall, in at least partial alignment with the internal element (21). In particular, the internal element (21) is collapsed on the placement tool (46) to decrease the cross sectional area thereof and help ease placement in the ventricle. Proximal and distal pins (25, 26) of the free wall leaf (23) of the internal element (21) engage proximal and distal hooks (34 and 35) on the pivoting tray (28) of placement tool (46) and are held in place by tension created by the convex positioning of the resilient free wall leaf (23) of the internal element (21).

Also shown is a guide wire (30) extending from the placement tool (46) through apertures (47) in the septal and free wall leaves (23, 24) of the internal element (21). In some embodiments, more than one guide wire (30) is used. Correspondingly, a plurality of apertures (47) may be present on the leaves (23, 24) of the internal element (21).

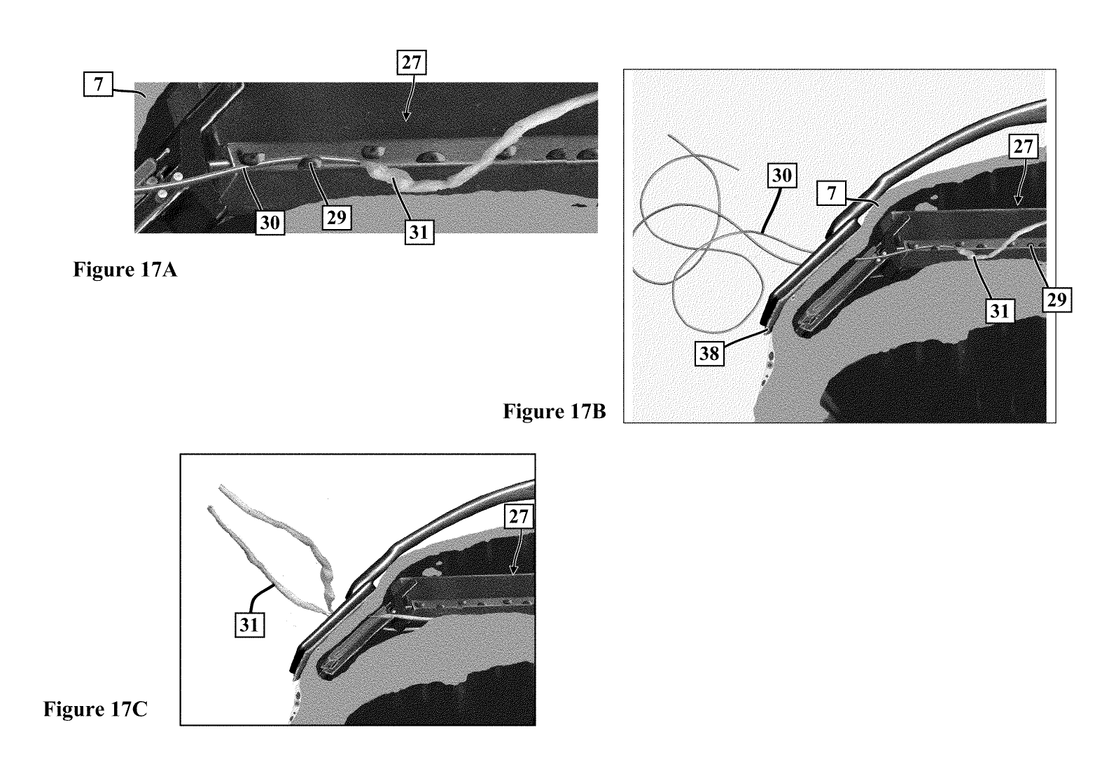

FIG. 12 shows that the inner jaw (27) of the placement tool (46) further includes a series of alternating guides (29) adjacent the pivoting tray (28) so as to secure the guide wire (30), but sufficiently spaced and smooth contoured so as not to interfere with free exit of a tow of unorganized fibers of fiber tows (31) (FIG. 17A) coupled to an end of the guide wire (30). An exemplary guide wire (30) has a diameter of approximately 0.035″ (i.e., ˜0.875 mm) with the degree of flexibility of the type commonly used in interventional radiology and cardiology. The unorganized fibers of fiber tows (31) are attached to the end of the guide wire (30) and are subsequently pulled into position, as described in greater detail below. The unorganized fibers of fiber tows (31) may optionally be the radial strands (3) of the septal splint (1). The alternating guides (29) may be any shape suitable for retaining the guide wire (30). For example, the alternating guides (29) may be knob-shaped and of a rigid metal or polymer, or they may comprise two opposing sinusoidal elastomeric strips.

Also shown is a hinge point (32) on which the tray (28) pivots and at least one spring-acting “straightener” mechanism, which keeps the tray in approximately 160 degree alignment, as shown. The pins-sliding-in-socket (33), with coiled compression socket spring (not shown), is one non-limiting example of the “straightener” mechanism; torsion springs at the hinge are a non-limiting alternative.

Features of the pivoting tray (28) include at least one point for retaining the internal element (21) in a collapsed manner during positioning the heart. For example, proximal (34) and distal (35) pin-hook and proximal (36) and distal (37) abutments of the placement tool (46) stabilize the internal element (21) position during unlocking of the placement tool (46).

The internal element (21) and/or external element (38) (FIG. 13) may also be padded with one or more layers of biocompatible surface materials such as textile fabrics and polymeric membranes. The internal element (21) and/or external element (38) may also allow for tissue ingrowth, including endothelialization, at their surfaces and may optionally be treated with structural modifications and or biologically active agents to promote tissue ingrowth.

FIG. 13 shows an embodiment of the outer jaw (39) which includes an open frame (40) mounted on an arm portion (41) of the placement tool (46). The angle of mounting is such that when the placement tool (46) closes and locks, the inclination of frame (40) on the outer jaw (39) approximates that of the tray (28) on the inner jaw (22) with full flexion of hinge pivot (32) against resistance of spring mechanism (33). An opening in the frame (not shown) allows visualization of the transparent part (42) of external element (38)—and, through the transparent part (42) of the external element (38)—also the epicardial surface of the heart to allow position adjustment for avoidance of coronary vessels. The open frame (40) may be rectangular, as shown, or any other shape that allows visualization of the external element (38). The transparent part (42) of the external element (38) may be made of polycarbonate but alternatively may be any other suitable transparent polymer or a glass. The transparent part (42) has at least one aperture (43) through which a guide wire (30) and then fiber tow (31) may be pushed and pulled during fixation. Both inner and outer surfaces of the aperture (43) are tapered or flared, and the channel thereof is inclined relative to the remainder of the transparent part (42) of the external element (38) to facilitate passage of guide wires (30). In the embodiment shown, the transparent part (42) of the external element (38) has two apertures (43) which correspond to the apertures (47) in the internal element (21).

The pad (44) of the external element (38) is configured to fit mound and support the transparent part (42). Pad (44) is generally soft, porous, biocompatible (e.g. PTFE felt or PET knit), and subject to ingrowth by and healing fixation to tissue such as epicardium.

It is understood that shapes in the plane of the frame-external element interface may be square, elliptical, circular, or any other shape in addition to the simple rectangular shape as shown. Correspondingly, the open frame (40) of the outer jaw (39) of the placement tool (46) may be square, elliptical, circular, rectangular (shown), or any other shape capable of holding the external element (38) and providing visual access to the transparent part (42).

FIGS. 14-19 show the steps for placement of the internal element (21) and external element (38) using the placement tool (46).

FIG. 14 (step one) shows the placement tool (46), with the internal element (21) mounted on the pivoting tray (28) of the inner jaw (27) as described above being advanced into the cavity of the right ventricle and the external element (38) mounted on the outer jaw (39). The inner jaw (27) traverses an incision in the base of the tricuspid leaflet's septal leaflet (not shown) and passes along the surface of the ventricular septum (9) behind papillary muscles and chordae tendinae (12) toward the junction of the right ventricular freewall (7) and the septum (9). The inset shows the distal end-pin (26) hooked securely in the distal tray hook (34); the proximal end-pin (25) is similarly secured in the proximal hook (34).

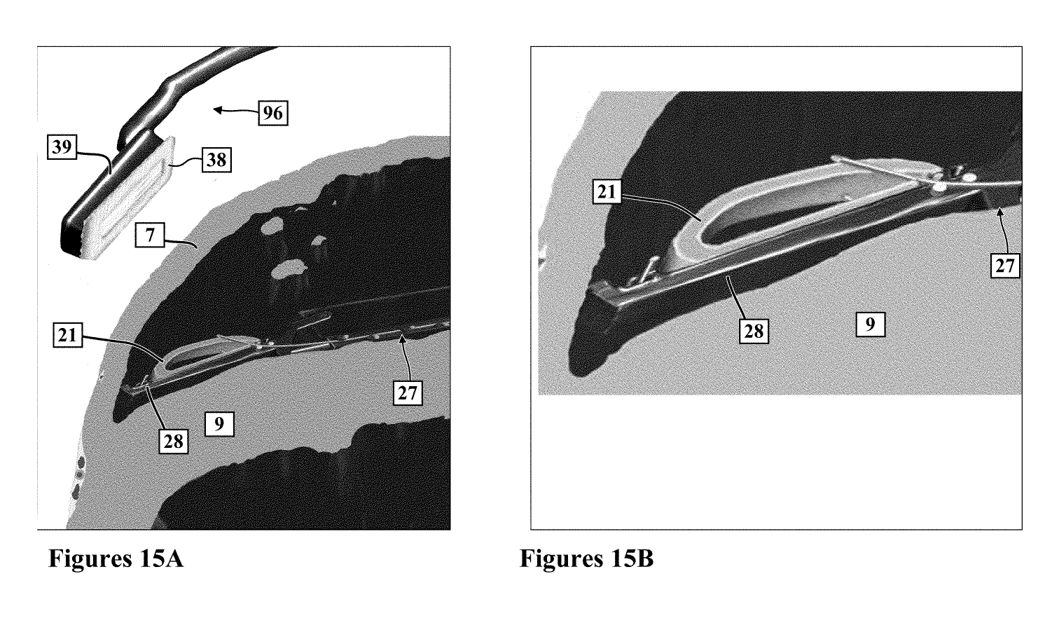

FIG. 15A (step two) shows the inner jaw (27) with the internal element (21) positioned at the junction of the free-wall (7) and ventricular septum (9), and being moved into alignment with the outer jaw (39) and the external element (38).

FIG. 15B shows a closer view of the relationship between the internal element (21) mounted on the tray (28) of the inner jaw (27), and the contiguous heart structures (7, 9).

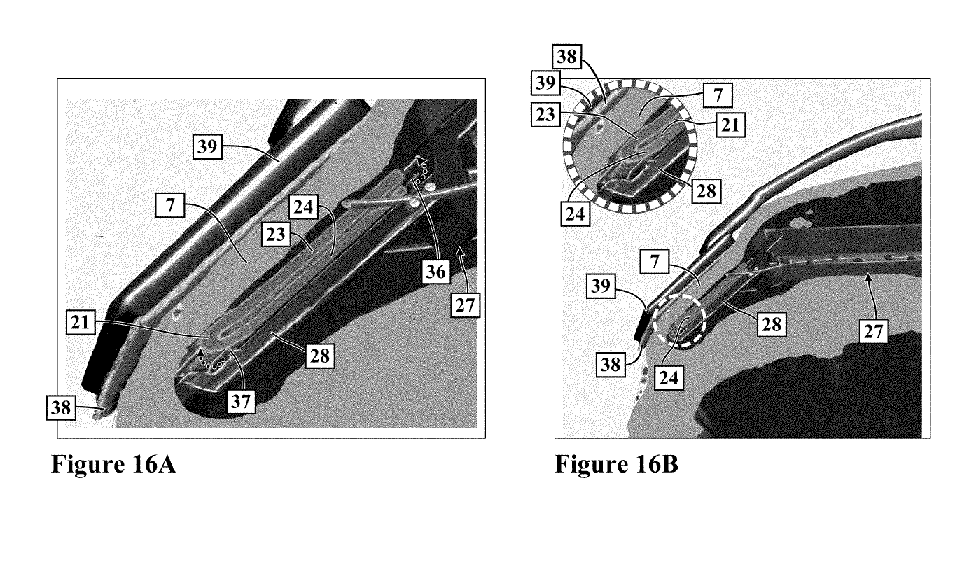

FIGS. 16A and 16B (step three) illustrate a compressive force delivered by the outer jaw (39) and the external element (38). That force is transmitted through the local region of the right ventricular free wall (7), which is resisted by the extension tray (28) and further compresses internal element (21), including its septal (24) and free wall (23) leaves. The compressed internal element (21) necessarily straightens from its prior convexity towards the free wall (7). And, the ends of internal element (21) strike the proximal and distal abutments (36, 37) causing proximal (25) and distal (26) endpins to dislocate from under proximal (35) and distal (34) hooks of extension tray (28), as indicated by the dotted lines.

FIGS. 17A and 17B (step four) show guide wires (30) pushed through the free wall of the right ventricle (7) and into the aligned apertures (43) (FIG. 13) of the external element (38). The tips of guide wires (30) then are grasped and pulled. Note that the fiber tows (31), being extremely flexible—far more so than the guide wires (30)—no longer remain in the guides (29).

FIG. 17C (step five) shows the two fiber tows (31) or radial strands (3) secured and the placement tool (46) still engaged and locked. The ends of the radial strands (3) may be secured by any methods known to the skilled artisan, such as by tying.

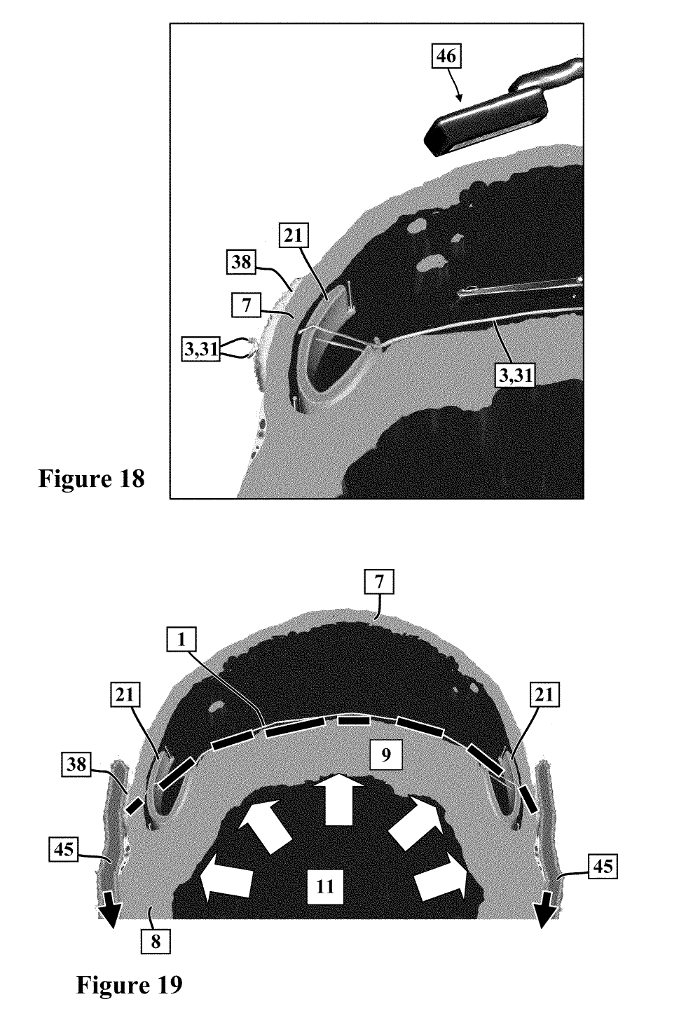

FIG. 18 (step six) shows the placement tool (46) unlocked and being withdrawn. This is repeated radially until all 6 to 15 pairs of radial strands (3) or tows (31), for example, and corresponding bolsters (48) are in place and anchored through the free wall (7).

FIG. 19 shows the cross-section of a left and right ventricle wherein the margins of the free-wall actuating mechanism (45) are linked by the septal splint (1) and the external and internal element (21, 38) of the bolsters (48) across the right ventricular free wall (7) to support the ventricular septum (9). Thus, the outward force of pressure in the left ventricular cavity (11), as indicated by the white arrows, is balanced as the active portion of the actuating mechanism (45) pushes in on the free wall of the left ventricle (8). Note that the line of force in this plane, represented as a dashed line, is essentially a circular arc—its lowest energy configuration—while the heavily vessel-bearing regions of the interventricular grooves have simply been displaced a few millimeters inward. Again, examples of free-wall actuating mechanisms are more fully described in U.S. Pat. No. 5,957,977 and U.S. Publication Nos. 2006/0155160 and 2006/0187550, each of which are incorporated by reference herein in their entirety. However, in brief, the devices described therein apply compressive force to the free wall of the left ventricle using various mechanical processes. In general, the devices are attached to the outside surface of the left ventricle and their shape is mechanically deformed to apply compressive forces to the outside surface of the heart.

Although the invention has been described in detail in the foregoing embodiments for the purpose of illustration, it is to be understood that such detail is solely for that purpose and that variations can be made therein by those skilled in the art without departing from the spirit and scope of the invention except as it may be described by the following claims.

What is claimed is:

1. A bolster comprising:
an external element defining an elongated body member including at least one aperture extending therethrough, the elongated body member being configured for placement adjacent an external surface of a cardiac wall; and
an internal element including a free wall leaf and a septal leaf joined together to define a body member, the free wall leaf including at least one aperture extending therethrough, the body member being configured for placement inside a cavity of the heart, wherein the external element and the internal element are configured for coupling to each other across the cardiac wall via the apertures.

2. The bolster of claim 1 wherein at least one of the external element or internal element is rigid.

3. The bolster of claim 1 wherein at least one of the external element or internal element is resilient.

4. The bolster of claim 3 wherein the body member includes at least one pin located at a distal or proximal end thereof.

5. The bolster of claim 1 wherein the elongated body member of the external element has a transparent portion.

6. The bolster of claim 1 wherein the elongated body member of the external element further comprises a pad secured thereto.

7. The bolster of claim 1 further including one or more rigid elements, compressive elements, and/or tensile elements for coupling the external element and internal element to each other across the cardiac wall via the apertures.

8. The bolster of claim 1 wherein at least one of the external element or internal element is capable of tissue ingrowth.

9. The bolster of claim 1 wherein the body member of the internal element is curved.

10. The bolster of claim 1 wherein the septal leaf includes at least one aperture extending therethrough.

11. A system for assisting the function of a natural heart comprising:
a septal splint configured for use in assisting the function of a natural heart; and
at least one bolster configured to secure the septal splint to a cardiac wall, the bolster comprising:
an external element defining an elongated body member including at least one aperture extending therethrough, the elongated body member being configured for placement adjacent an external surface of the cardiac wall; and
an internal element including a free wall leaf and a septal leaf joined together to define a body member, the free wall leaf including at least one aperture extending therethrough, the body member being configured for placement inside a cavity of the heart, wherein the external element and the internal element are configured for coupling to each other across the cardiac wall via the apertures.

12. The system of claim 11 further comprising a free-wall actuating mechanism that is configured to be coupled to the septal splint via the bolster.

13. The system of claim 11 wherein the at least one bolster includes a plurality of bolsters configured to secure the septal splint to a cardiac wall.

14. The system of claim 11 wherein at least one of the external element or internal element is rigid.

15. The system of claim 11 wherein at least one of the external element or internal element is resilient.

16. The bolster of claim 11 further including one or more rigid elements, compressive elements, and/or tensile elements for coupling the external element and internal element to each other across the cardiac wall via the apertures.

17. A method for assisting the function of a natural heart comprising:
securing at least one bolster to a cardiac wall, the bolster comprising:
an external element defining an elongated body member including at least one aperture extending therethrough, the elongated body member being configured for placement adjacent an external surface of the cardiac wall; and
an internal element including a free wall leaf and a septal leaf joined together to define a body member, the free wall leaf including at least one aperture extending therethrough, the body member being configured for placement inside a cavity of the heart, wherein the external element and the internal element are configured for coupling to each other across the cardiac wall via the apertures;
securing a septal splint to the cardiac wall via the bolster, the septal splint being situated in the right ventricle of the heart; and
coupling a free-wall actuating mechanism to the septal splint via the bolster, the free-wall actuating mechanism positioned over the left ventricle of the heart.

18. The method of claim 17 wherein securing at least one bolster to a cardiac wall includes securing a plurality of bolsters to the cardiac wall and wherein securing a septal splint to the cardiac wall via the bolster includes securing a septal splint to the cardiac wall via the plurality of bolsters.

19. The method of claim 17 further comprising actuating the free-wall actuating mechanism.

20. The method of claim 17 wherein the bolster further includes one or more rigid elements, compressive elements, and/or tensile elements for coupling the external element and internal element to each other across the cardiac wall via the apertures.
